(19) United States
(12) Patent Application Publication
(10) Pub. No.: US 2008/0311680 A1
(43) Pub. Date: Dec. 18, 2008

(54) Method for detecting high antigen concentration and device therefor

(76) Inventor: John Chiu, Torrance, CA (US)

Correspondence Address: SAM CHEN, 7F-1, 293, ROOSEVELT ROAD, SEC 3, TAIPEI, TW

(21) Appl. No.: 11/762,781

(22) Filed: Jun. 14, 2007

(51) Int. Cl. G01N 33/536; C12M 1/00

(52) U.S. Cl. 436/536; 422/188

(57) ABSTRACT

A method for detecting high antigen concentration is disclosed. The method enables the mobile-phase antibody, in the presence of excessive amount of antigen, to form the antibody-antigen-antibody sandwich with the immobilized solid-phase antibody effectively in a rapid lateral flow chromatographic immunoassay. The mobile-phase and/or immobilized solid-phase antibody are treated with soluble coatings to generate a delaying mechanism, so that antigen-antibody binding occurs only when both phases of antibodies and antigen are in very close proximity. A user friendly immunoassay device with a sample over-flow mechanism also facilitates such antigen-antibody binding.

### BACKGROUND OF THE INVENTION

1. Field of Invention

The invention relates to lateral flow chromatographic immunoassay methods and devices and more particularly to a method for detecting high antigen concentration by treating mobile phase antibody and/or immobilized solid-phase antibody with soluble coatings to generate a delaying mechanism, so that antigen-antibody binding occurs only when both phases of antibodies and antigen are in very close proximity. The invention also relates to a user friendly device based on this method with a sample over-flow mechanism for facilitating such antigen-antibody binding.

2. Description of Related Art

Lateral flow chromatographic immunoassay method and device have been described extensively. (Gordon and Pugh, U.S. Pat. No. 4,956,302; H. Buck, et. al., WO 90/06511; T Wang, U.S. Pat. No. 6,764,825 BI; W. Brown, et. al., U.S. Pat. No. 5,008,080; Kuo and Meritt, US 06183972, EP 00987551A3). This technique has been commercialized for the productions of easy-to-use rapid diagnostic tests, such as Clearblue One-Step Pregnancy Test in 1988 (EP 291194; EP 560411).

Typical One-Step Test consists of four continuous domains in line. They are (1) sample domain for the addition of sample solution, (2) mobile-phase domain containing the mobile “color conjugate” made from conjugation between “detection antibody” and “color marker,” (3) solid-phase domain containing immobilized “capture antibody,” and (4) absorption domain containing an absorber. To do the testing, sample is added to the sample domain first. The sample solution then flows to the mobile-phase domain where the antigen (the analyte to be detected) in the sample binds the “color conjugate” through the “detection antibody” portion and form “antigen-color conjugate complex.” This color complex then migrates through the solid-phase domain chromatographically, and is caught by the “capture antibody” through the antigen portion of the color complex, forming a sandwich of “capture antibody-antigen-color conjugate complex.” Now, since the “capture antibody” is immobilized on the solid-phase domain, the whole sandwich also becomes immobilized on the solid-phase domain, thus creating a “visible color zone” in the solid-phase domain. On the other hand, if there is no antigen in the sample, no such sandwich can be formed, thus creating no color zone. Meanwhile, all non-captured materials and solution contents continue to migrate further and be absorbed by absorber in the absorption domain.

This kind of test requires the end user to simply add the sample and then observe the result a few minutes later; it is called One-Step Test. Since such rapid and easy-to-use test is user friendly, it is very popular in both the professional and consumer markets nowadays. However, when the sample contains excessive amounts of antigen (the analyte to be detected) the final “visible color zone” may become very weak or invisible, a phenomenon known as the Hook Effect, i.e., the dose-response curve of antigen concentration verses signal intensity somewhat resembles a hook. The reason for this phenomenon to occur is detailed below. High concentration of antigens in the sample first react with the “detection antibodies” of the “color conjugate” in the mobile-phase domain and saturate all the binding sites of the “detection antibodies” immediately. That is, all of the “color conjugates” now have changed into the “antigen-color conjugate complex.” Next, the excessive amounts of free antigen quickly migrate chromatographically into the solid-phase domain to react with the immobilized “capture antibodies” and saturate all the binding sites of the “capture antibodies.” Therefore, when the “antigen-color conjugate complex” migrates into the solid-phase domain later on, no sandwich (“visible color zone”) can be formed, because all the antigen binding sites of the “capture antibodies” have been pre-saturated by the excessive amounts of free antigens moved in earlier.

The reason for the free antigens to be able to move ahead of the “antigen-color conjugate complex” and to bind the immobilized “capture antibodies” first is due to the fact that the physical size and mass of the antigen are much smaller then those of color complexes, so they have a higher diffusion rate. Moreover, the lateral flow chromatographic movement in the solid-phase domain generates the partition effect.

Efforts have been made to minimize the Hook Effect so as to better detect the present of exceeding high concentration of antigen in One-Step immunoassay. They are: (1) Increase the zone number of “capture antibody” to broaden the antigen detection range (C. Lee, et. al., J. Clinical Ligand Assay, 2004/2005; 27:262; Kuo and Meritt, U.S. Pat. No. 6,183,972 and EP 00987551A3), (2) Let the sample first reacts with the solid-phase “capture antibody” and then, after incubation and washing step, reacts with the mobile-phase “detection antibody,” changing it to a Two-Step immunoassay (Khosravi and Diamandis, Clin. Chem. 1987, 33:1994; G. Bodor, et. al., Clin. Chem. 1989; 35:1262), and (3) Dilute the sample or increase the concentrations of antibodies (M. Khosravi, Clin. Chem. 1990; 36:1169). However, all these improvements do not solve the problem of Hook Effect substantially, but do make the One-Step Test more expensive, more difficult to make, and more complicate to perform and interpret the results. For example, Kuo and Meritt (U.S. Pat. No. 6,183,972 and EP 00987551A3) teaches a method, similar to that taught by G. Lee, et. al. (J. Clinical Ligand Assay, 2004/2005; 27:262), which requires at least one extra zone of “capture antibody” in the solid-phase domain, and for result interpretation, a monotonous dose-response standard curve should be established beforehand, a reflectance spectrometer is required for result signal detection, and data calculations are required. Finally, the detection range covers not greater than 70-fold of concentration variation.

Since the amount of the analyte in the sample is unknown before the testing, high concentrations of analyte leading to false negative results may have serious medical, economical, or social impacts and consequences. Therefore, immunoassay with broad analyte concentration detection range is very useful and important.

Thus, it is desirable to provide a novel method capable of employing One-Step Diagnostic Test that can make qualitative detection of analytes at high concentrations will be very helpful to the users.

### SUMMARY OF THE INVENTION

It is therefore one object of the invention to provide a method for detecting high antigen concentration. The method enables the mobile-phase antibody, in the presence of excessive amount of antigen, to form the antibody-antigen-antibody sandwich with the immobilized solid-phase antibody effectively in a rapid lateral flow chromatographic immunoassay. The mobile-phase and/or immobilized solid-phase antibody are treated with soluble coatings to generate a delaying mechanism, so that antigen-antibody binding occurs only when both phases of antibodies and antigen are in very close proximity.

It is another object of the invention to provide an immunoassay device for detecting high antigen concentration wherein a sample over-flow mechanism is employed to facilitate such antigen-antibody binding.

The above and other objects, features and advantages of the invention will become apparent from the following detailed description taken with the accompanying drawings.

### BRIEF DESCRIPTION OF THE DRAWINGS

FIG. 1 schematically depicts four continuous domains of a One-Step immunoassay where color conjugates have been coated with a predetermined amount of coating materials according to the invention;

FIG. 2 is a view similar to FIG. 1 where sample containing high concentration of antigen (i.e., the analyte to be detected) is added to first domain at the start of the One-Step immunoassay;

FIG. 3 is a view similar to FIG. 2 where substantially no color conjugates in second domain can bind with antigens migrated from first domain;

FIG. 4 is a view similar to FIG. 2 where a partition effect has differentiated molecules into three zones of free antigen, coated color conjugate, and antigen-color conjugate-complex in third domain;

FIG. 5 is a view similar to FIG. 2 where after quickly saturating all color conjugates in second domain by high concentration of antigen which in turn moves forward quickly to saturate all capture antibodies in third domain for forming a plurality of sandwiches of color conjugate-antigen-capture antibody;

FIG. 6A is a side elevational view of a stick shaped immunoassay device according to the invention with a test strip secured therein;

FIG. 6B is a view similar to FIG. 6A where the case has been removed and then put on the handle prior to testing; and

FIG. 7 shows four continuous domains of a test strip of the immunoassay device in side elevation for schematically depicting a flooding mechanism of a sample solution added to the test strip according to the invention.

### DETAILED DESCRIPTION OF THE INVENTION

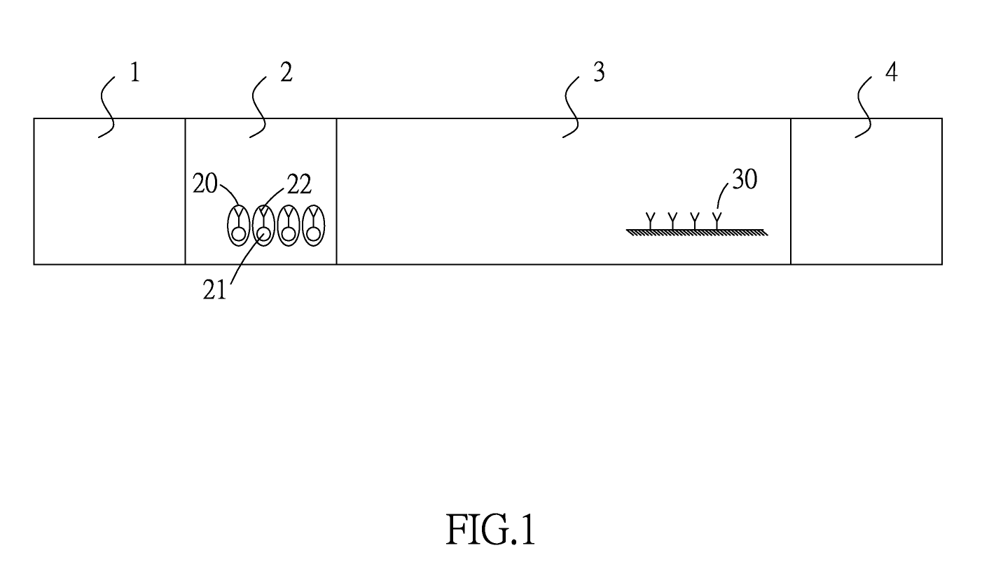

Referring to FIG. 1, a method for detecting high antigen concentration in accordance with the invention is embodied in a One-Step immunoassay which comprises four continuous domains. These four domains are mounted over a single solid support which allows the liquid sample to be applied onto the first domain 1, continues to flow and migrate chromatographically through the second and third domains 2, 3 in a lateral flow manner, and ends up with being absorbed in the fourth domain 4.

The first domain 1 is a porous solid material such as paper, glass fiber, resin, or porous plastic. This porous solid material should be treated and then dried with a buffer system which is able to pre-condition the sample solution so that when the antigens (i.e., the analytes to be detected) migrate into the second and third domains 2, 3 it can bind with antibodies effectively in a favorable condition in second and third domains 2, 3. Moreover, this porous solid material is able to filtrate and/or remove particulates, viscous, interfering, and unwanted materials from the sample, so that the binding of antigen 11 and antibody 22 or 30 will be more efficient. Since the first domain 1 is for the addition of sample, it is a sample domain 1.

The second domain 2 is a porous soft material such as glass fiber, membranes, resin, or porous plastic. This domain is pre-treated, either by spotted on or merged into, with color conjugate and then dried in air or in vacuum. The color conjugate is made from conjugation between detection antibody 22 and color marker 21. The detection antibody 22 is an antibody which can bind antigen 11 specifically, effectively, and quickly. The color marker 21 is a visible marker or label such as colloidal gold, polystyrene bead, or dye particles. The color marker 21 should not interact or bind with materials in the sample or in the buffer system, and it should be able to form conjugation with the detection antibody 22. Furthermore, before embedding onto the second domain 2, the color conjugate should be added with a buffer containing predetermined amount of coating materials, so that when the color conjugate is re-hydrated by the sample solution, it will become mobile and can migrate into the next domain, yet, the antibody 22 on the color conjugate will not interact with the antigen 11 in the sample immediately and entirely. In a sense, the coating materials provide a “delaying mechanism” for the binding between the antigen 11 in the sample and the antibody 22 on the color conjugate to occur fully and immediately, but until a predetermined time (achieved by dissolution of the coating materials during migration) has come. Since the second domain 2 contains migratory conjugates, it is a mobile-phase domain 2.

The third domain 3 is a porous matrix such as nitrocellulose membrane, resin, or porous plastic, which allows molecules to migrate through it chromatographically. During chromatographic movement, molecules will be partitioned into different migration zones. At a predetermined section of the third domain 3, the capture antibody 30 is immobilized on it either by chemical means or by physical means. The capture antibody 30 is able to bind the antigen 11 at a site other than the site bound by the detection antibody 22. If the antigen 11 has repeated one kind of binding site, then both the detection antibody 22 and the capture antibody 30 are of the same kind can be used under certain conditions. When in need, the capture antibody 30 is also coated with a predetermined amount of coating materials to provide another “delaying mechanism”. In this case, the first arrival of small antigen molecules 11 will not be able to saturate all the antigen binding sites of the capture antibody 30 fully, because the coating materials are still covering some of the capture antibody 30. It can be pre-arranged so that the coating materials are fully dissolved and removed, however, when the large color conjugate molecules are moving into the vicinity of the immobilized capture antibody 30. Since the third domain 3 contains immobilized antibody 30, it is a solid-phase domain 3.

The fourth domain 4 is an absorption matrix such as paper or hydrophilic porous plastic. This matrix can absorb the entire amount of sample liquid and facilitate the migration of antigen 11, color conjugate, antigen-color conjugate complex, and all other unbounded sample materials from the first domain 1 to the fourth domain 4. Since the fourth domain 4 contains absorbent pad, it is an absorption domain 4.

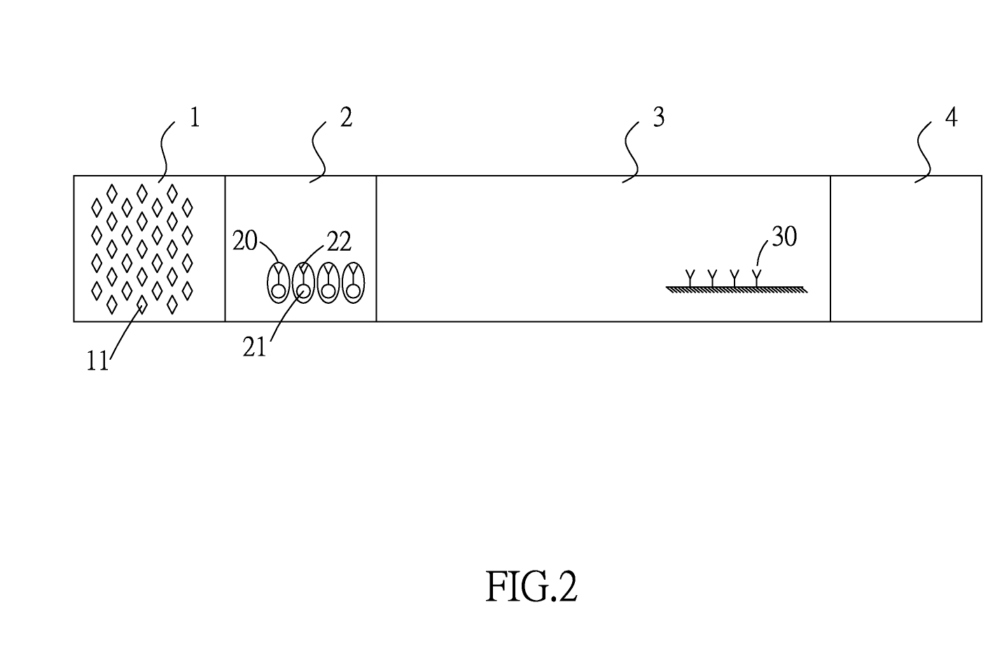

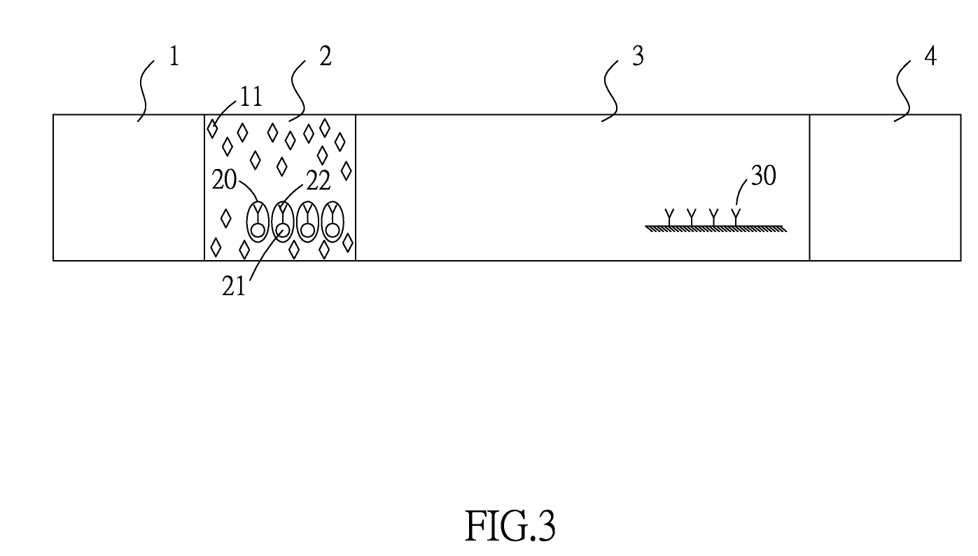

The method for detecting high antigen concentration in accordance with the invention is illustrated by referring to FIGS. 2 to 5. In FIG. 2, a high concentration of liquid sample of urine, serum, whole blood, spinal fluid, or an unknown solution with or without antigen (i.e., the analyte to be detected) 11 is added to the first domain 1 at the start of the One-Step immunoassay. In the second domain 2 of FIG. 3, after the filtration by the sample pad matrix, the sample solution moves into the second mobile-phase domain 2 and re-hydrate the color conjugate. If the color conjugate is not pre-coated with coating materials, all of the detection antibodies 30 of the color conjugate will immediately bind and be saturated by the excessive amount of antigen 11 in the sample when the sample contains high concentration of antigen 11, and form the undesired antigen-color conjugate complex entirely as done by conventional methods. Advantageously, as contemplated by the invention, the color conjugate is pre-coated with a predetermined amount of coating materials when antigen concentration in sample is high. Hence, only a very few amount of coated color conjugates 20 becomes antigen-color conjugate complex while most others remain as the desired coated color conjugates 20.

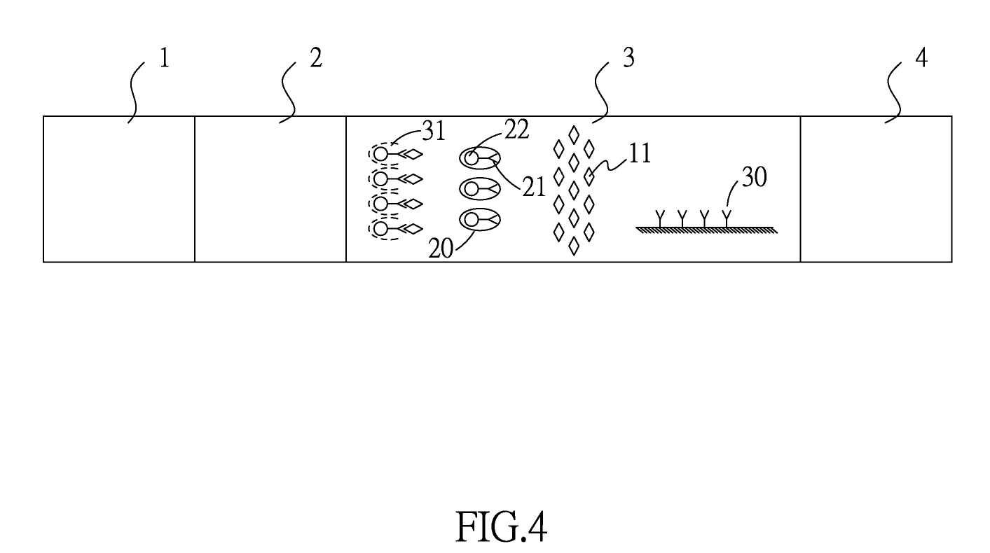

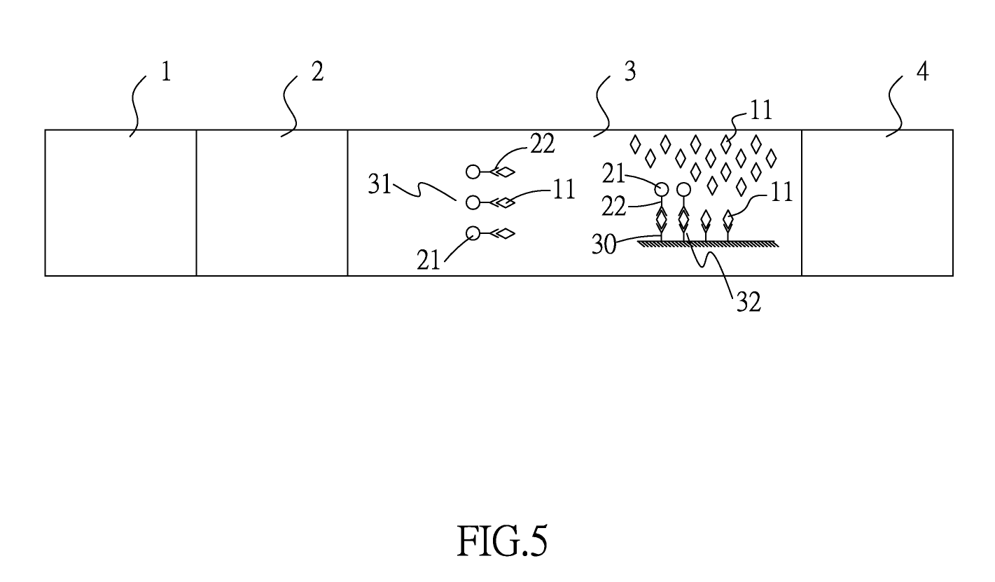

In the third domain 3 of FIGS. 4 and 5, this liquid mixture then migrates into the third solid-phase domain 3 chromatographically. The partition effect can separate molecules into three different zones. The small and unbound antigen molecules 11 migrate rapidly in the front, followed by the coated color conjugate 20, then the antigen-color conjugate complex 31.

In the presence of exceedingly high concentration of antigen 11 in the sample as done by conventional methods, the excessive free antigens 11 in front can quickly saturate all antigen binding sites of the capture antibody 30 to form the antigen-capture antibody complex immediately and fully. Therefore, if the color conjugate had not been pre-coated as done by conventional methods, all color conjugates would have been changed to the undesired antigen-color conjugate complex in the second domain 2. When these complexes move to the capture antibody regions in the third domain 3, since all capture antibodies 30 will be converted into the antigen-capture antibody complex before the arrival of the antigen-color conjugate, no color conjugate-antigen-capture antibody sandwich can be formed, a false negative result is obtained by the conventional methods.

Advantageously, as contemplated by the invention, coating can prevent physical contact until the coating materials have been dissolved or removed, if the color conjugate had been pre-coated, when the coated color conjugate 20 and antigen-color conjugate move into the capture antibody regions 30, only the coated color conjugate 20 may be de-coated to the unbound color conjugate and then may bind to the antigen 11 of the antigen-capture antibody complex, thus forming the sandwich of color conjugate-antigen-capture antibody 32. If the capture antibody 30 is also pre-coated with a predetermined amount of coating materials then more color sandwich 32 can be formed. Since the capture antibody 30 is immobilized in the solid-phase domain 3, a visible color zone appears in the capture antibody region (i.e., in the third domain 3), a positive result signal is obtained. Conversely, if no antigen 11 is present in the sample, the bridge connecting the color conjugate and the capture antibody 30 does not exist, thus no color sandwich 32 can be formed, a true negative result is obtained.

For the fourth domain 4, all movable liquid and molecules will be absorbed by the absorbent pad in this domain. The absorption capacity of the absorbent pad is made large enough to prevent back flow of liquid for at least two hours.

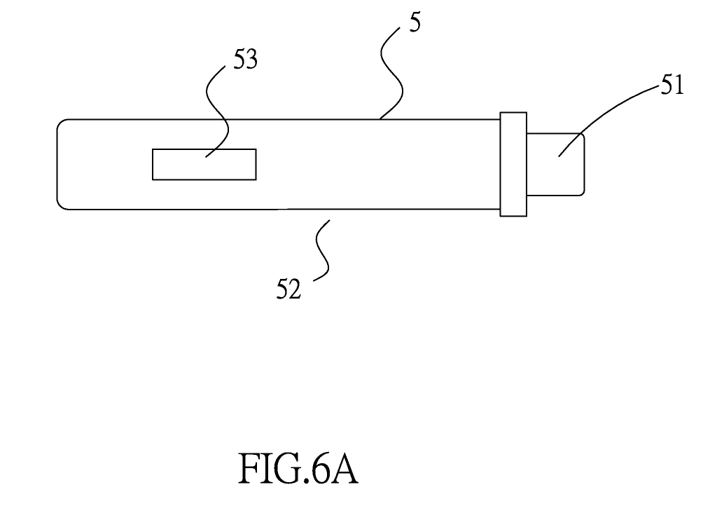

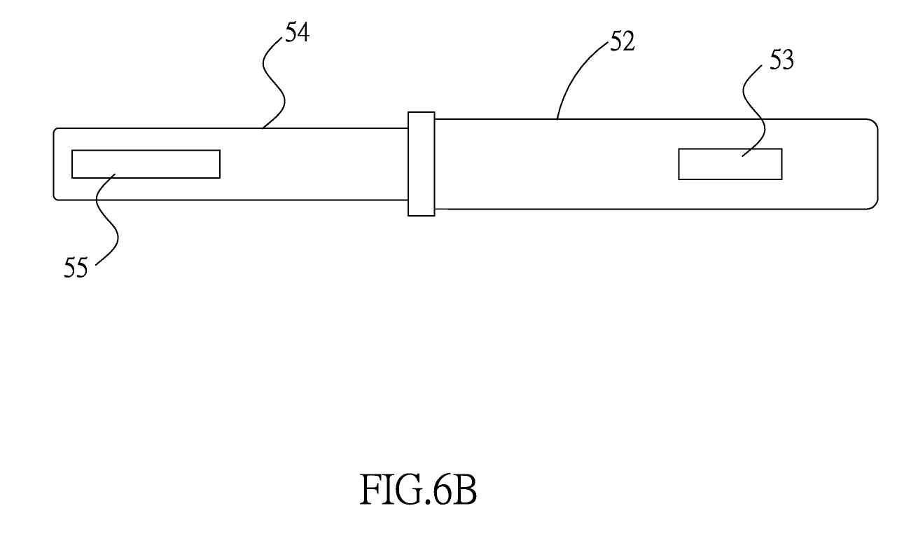

Referring to FIGS. 6A and 6B, a stick shaped immunoassay device 5 for implementing the above high antigen concentration detection method according to the invention is shown. The immunoassay device 5 is enclosed by a case 52 with a handle 51 projecting rearward. The case 52 comprises a rectangular window 53. A slot 55 is formed on a front portion of a plastic stick 54 which is exposed after removing the case 52. Also, a test strip (not shown) is secured within the see-through stick 54.

When in use, the case 52 is removed and then put on the handle 51 so as to make the handle 51 longer for better handling. Next, a liquid sample can be applied directly onto the slot 55. After use, the case 52 is put back to the original position and the test result can be seen through the window 53 of the case 52. This user friendly immunoassay device 5 can utilize sample over-flow mechanism to facilitate correct qualitative detection of the antigen when the antigen concentration is high.

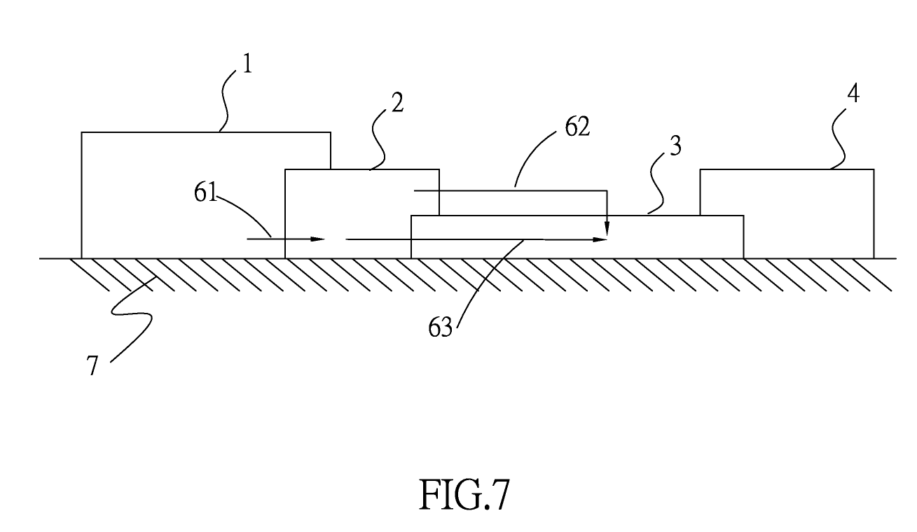

Referring to FIG. 7 in conjunction with FIGS. 6A and 6B, a flooding mechanism of a sample solution added to the test strip according to the invention is shown. The main body of this device 5 is the test strip secured to a support 7 thereunder with four continuous domains 1, 2, 3 and 4 lying on top of it in line. The first domain 1 is a porous solid material pre-treated with buffer. This is the sample domain 1 for sample application. The second domain 2 is a porous soft material containing the color conjugate with a predetermined amount of coating materials. This is the mobile-phase domain 2, on one end it is inserted underneath the first domain 1 and on the other end it is laying on top of the third domain 3. The third domain 3 is a membrane, which contains the immobilized capture antibody and which allows chromatographic migration of liquids. This is the solid-phase domain 3, on one end it is inserted underneath the second domain 2, on the other end it is laying underneath the fourth domain 4. The fourth domain 4 is an absorbent pad which can draw the sample liquid from the first domain 1 toward it and also is able to take up the entire sample liquid amount. This is the absorption domain 4, on one end it is laying on top of the third domain 3.

Overall, the test strip is housed in a plastic test stick 54 having a window 53. The front portion of the test stick 54 has a slot 55 aligned with the first domain 1 for sample addition, by cutting through a stream of the sample, dipping into the sample, or adding sample onto it. The slot 55 has a dimension that can collect and retain sufficient amount of sample liquid for testing in half a second when this slot 55 is in contact with the sample liquid. This high efficiency of sample solution pick-up is facilitated by the surface tension and capillary effects on the slot 55. The slot 55 is located right next to the second domain 2 so that after the addition of sample to the first domain 1 the sample not only reaches the second domain 2 immediately but also creates a flooding over the third domain 3. In this case, some of the sample solution with coated color conjugate 20 can flow on top of the third domain 3 (as indicated by arrow 62 in FIG. 7) without the effect of partition from the third domain 3, and reach the immobilized capture antibody 30 almost as quickly as the free and unbound antigen 11. Therefore, some of the fast reaching coated color conjugate 20 will be able to react with antigen-capture antibody complex and form the visible color sandwich 32, showing a positive result in the presence of exceedingly high amount of antigen 11. Moreover, due to this rapid flooding mechanism, test result can appear in as soon as 10 seconds. Meanwhile, certain amount of sample solution migrates chromatographically through the inside of the third domain 3 (as indicated by arrow 63 in FIG. 7). As such, the free and unbound antigen 11 (as indicated by arrow 61 in FIG. 7) will reach the capture antibody 30 region faster than the coated color conjugate 20. These non-over-flow coated color antibody has less chance to form sandwich than the over-flow coated color antibody because it may have been de-coated somewhat during the migration in the solid-phase domain 3. Thereafter, all unbound materials and solution are absorbed in the fourth absorption domain 4.

### EXAMPLE 1

This example provides a control to demonstrate that a regular One-Step lateral flow chromatography immunoassay is sufficient to detect antigen at a certain low range of concentration, but it gives false negative result when the concentration has been increased by 100,000 folds.

The test strip at 4 mm×60 mm for the detection of HCG was prepared (Vanguard Biomedical Corporation, USA). The sample pad filter paper (Schleicher & Schuell, Germany) at 4 mm×12 mm was treated with 50 mM Tris buffer saline with 0.1% Tween 20, pH 8.3 and air dried at room temperature overnight, then dried at 45° C. with blowing air in an oven for one hour. The conjugate pad glass fiber at 4 mm×6 mm was treated with 20 μl of conjugate, OD at 530 nm=1.5, made from 40 nm colloidal gold and a monoclonal anti-beta-HCG antibody (Medix Biochemica, Finland) and dried in a lyophilizer at 25° C. for 5 hours. The nitrocellulose membrane at 4 mm×25 mm (Sartorius, Germany) was spotted with 10 ng of another monoclonal anti-beta-HCG antibody (Medix Biochemica, Finland) in 10 mM Tris buffered saline, pH 8.3 at the center region of the nitrocellulose and air dried at room temperature overnight. The absorption domain is an absorption paper at 4 mm×19 mm (Schleicher & Schuell, Germany). To assemble the test strip, a plastic strip (G & L, USA) at 4 mm×60 mm with double adhesive was attached with the membrane at about the center location. The absorption paper was attached to one end of the plastic strip, with a slight overlap with the membrane. The conjugate pad was attached to the other end of the plastic strip, with slight overlap with the membrane, followed by another attachment of the sample pad, with slight overlap with the conjugate pad. Finally, the test strip was assembles in a One-Step test cassette (Vanguard Biomedical Corporation, USA) before testing.

The test cassettes were added with 0.2 ml of non-pregnant woman's urine or 50 mM Phosphate buffered saline, pH 8.0 (PBS) containing 0.1% bovine serum albumin (BSA). Both samples showed no visible color signals, the negative results.

Other test cassettes were added with 0.2 ml of early pregnant woman's urine (within one week of her missed period) or 20 ml U/ml of HCG (First IRP from the WHO) in PBS-BSA. Both showed weak visible color signals, the positive results.

More test cassettes were added with 0.2 ml of pregnant woman's urine (two weeks after her missed period) or 1,000 mIU/ml of HCG (First IRP from the WHO) in PBS-BSA. Both showed strong visible color signals, the positive results.

Some test cassettes were added with 0.2 ml of three-month pregnant woman's urine or 2,000 IU/ml of HCG (prepared from acetone powder and calibrated with First IRP from the WHO) in PBS-BSA. Both showed no visible color signals in 10 minutes and then showed very faint signals after 30 minutes, indicating false negative results.

### EXAMPLE 2

This example provides the evidence to demonstrate that the invention is able to detect antigen over a range of 100,000-fold in concentration change, thus enables the detection of exceeding high concentration of antigen in the sample.

The test strip and test cassette preparations were the same as those described in Example 1, excepting that the buffer system of color conjugate also included certain predetermined coating materials such as sucrose, glycine, polyethylene glycol, glycerol, and surfactants to create a delaying mechanism.

All results were in similar with those shown in Example 1, excepting that when samples of three-month pregnant woman's urine and 2,000 IU/ml of HCG were used, both showed clear visible color signals in 5 minutes, revealing true positive results.

### EXAMPLE 3

This example shows that the test device of the invention with the sample over-flow mechanism not only can accommodate the test strip made with the invention method but also enhance the effect of this method. All together, this device makes the detection of antigen with the immunodiagnostic kit very easy to use and also quicker in getting the result.

Test strips identical to those in Example 2 were used. Instead of regular test cassettes, the test devices of the invention (FIGS. 6A and 6B) were used to house the test strips.

When in testing, the test stick was first removed from the case and then the handle was inserted into the case, thus making an extension to the handle. For sample addition, the tip of the test stick was cutting through a stream of sample solution for half of a second, thus allowing the sample to wet the slot of the test stick. In so doing, the entire movement of coated color conjugate can be seen through the see-through housing of the test stick. The same samples in Example 2 were used, and the same sample identifications (test results) in Example 2 were shown. In particular, visible color signals in the capture antibody region could be seen as soon as 10 seconds for all positive samples.

### ADVANTAGES OF THE INVENTION

Exceedingly high concentration of antigen (i.e., the analyte to be detected) can saturate all antibodies in the immunoassay system rapidly and prevent the formation of a complex of antibody-antigen-antibody sandwich, an essential part of a visible signal, leading to a false negative test result of the assay. The invention teaches a simple and easy method, a “delaying mechanism”, to enable the positive qualitative identification of the antigen in high concentration by pre-treating both the detection and/or capture antibodies with predetermined coating materials. In so doing, the antigen detection range can span over 100,000 folds.

The invention also provides an innovative immunoassay device with a flooding mechanism to facilitate the positive detection of ultra high concentration of antigen. Furthermore, this device can take up sufficient amount of the sample in a fraction of one second when this device is in contact with the sample solution. Therefore, the end user can use this device to cut through a stream of urine quickly then observe the progress of the reaction through the see-through test strip housing, and reads the test result as soon as 10 seconds through the window of the stick case. At the end, this device can be stored in a purse, pocket, or small container.

Unlike prior art, the invention solves the problem of Hook Effect leading to the false negative result in the qualitative detection almost entirely by increasing the detection range up to 100,000 folds, and, other than adding coating materials to the antibody buffers, the invention does not require extra manufacturing procedure, production cost, sample pre-treatment step, serial sample dilution step, and test result interpretation step to the now existing simple, easy, rapid, inexpensive, and user friendly One-Step Immunodiagnostic Test.

While the invention herein disclosed has been described by means of specific embodiments, numerous modifications could be made thereto by those skilled in the art without departing from the scope and spirit of the invention set forth in the claims.

What is claimed is:

1. A method for detecting high concentration of antigens of a test strip including a sample domain, a mobile-phase domain filled with color conjugates including a detection antibody and a color marker bound together wherein the color conjugates are coated with a predetermined amount of soluble coating materials to form a plurality of coated color conjugates, a solid-phase domain filled with immobilized capture antibodies, and an absorption domain, comprising the steps of:
(a) adding a liquid sample including a plurality of antigens onto the sample domain;
(b) migrating the antigens through the mobile-phase domain to the solid-phase domain substantially without binding with the coated color conjugates and migrating the coated color conjugates to the solid-phase domain;
(c) solving the coated color conjugates to bind with the antigens to form a plurality of antigen-color conjugate complexes;
(d) binding the antigen-color conjugate complexes with the capture antibodies to form a plurality of immobilized sandwiches of color conjugate-antigen-capture antibody; and
(e) causing the absorption domain to absorb the remaining unbound materials of the liquid sample migrating thereto.

2. The method of claim 1, wherein the capture antibodies are coated with a predetermined amount of soluble coating materials to form a plurality of coated capture antibodies.

3. An immunoassay device comprising:
a removable case having a surface window;
a plastic stick enclosed by the case and having a forward slot;
a test strip fastened in the stick and including a sample domain, a mobile-phase domain partially overlapped with the sample domain and being filled with color conjugates including a detection antibody and a color marker bound together wherein the color conjugates are coated with a predetermined amount of soluble coating materials to form a plurality of coated color conjugates, a solid-phase domain partially overlapped with the mobile-phase domain and being filled with immobilized capture antibodies, and an absorption domain partially overlapped with the solid-phase domain; and
a handle projecting rearward from the stick and being adapted to securely insert into the removed case in an operative position,
wherein in response to adding a liquid sample including a plurality of antigens onto the sample domain through the slot and migrating the antigens to the mobile-phase domain, a portion of the liquid sample continues to migrate to the solid-phase domain chromatographically, and the remaining portion thereof is adapted to quickly flow on a top of the solid-phase domain to give the coated color conjugates a possibility of forming a plurality of sandwiches with the capture antibodies.
